(19) United States
(12) Patent Application Publication
(10) Pub. No.: US 2015/0313891 A1
(43) Pub. Date: Nov. 5, 2015

(54) NEURONAL NICOTINIC AGONISTS AND METHODS OF USE

(71) Applicant: AbbVie Inc., North Chicago, IL (US)

(72) Inventor: Michael W. Decker, Mundelein, IL (US)

(21) Appl. No.: 14/702,308

(22) Filed: May 1, 2015

Related U.S. Application Data
(60) Provisional application No. 61/988,019, filed on May 2, 2014.

(51) Int. Cl. A61K 31/4748; A61K 31/439

(52) U.S. Cl. CPC A61K 31/4748; A61K 31/439

(57) ABSTRACT

An embodiment relates to a selective agonist of neuronal nicotinic acetylcholine receptor α7 subtype, a pharmaceutically suitable salt, prodrug, or a metabolite thereof, for the prevention and treatment of diseases and conditions that are mediated by nicotinic acetylcholine receptors, and methods of use thereof. Another embodiment is a method of administering a pharmaceutically effective amount of a selective agonist of neuronal nicotinic acetylcholine receptor α7 subtype, or a pharmaceutically suitable salt, prodrug, or a metabolite thereof, to a mammal in need thereof.

### RELATED APPLICATIONS

This application claims the benefit of priority to U.S. provisional patent application No. 61/988,019 filed on May 2, 2014. The contents of the above-mentioned priority application is hereby incorporated by reference in its entirety.

### STATEMENT REGARDING FEDERALLY SPONSORED RESEARCH OR DEVELOPMENT

Not applicable.

### FIELD OF THE INVENTION

It has been found that α7-nAChR agonists or α7-nAChR positive allosteric modulators may be used in the treatment, prevention or delay of progression of dyskinesia associated with dopamine agonist therapy in Parkinson's disease (PD). In particular, it has been found that α7-nAChR agonists or α7-nAChR positive allosteric modulators may be used in the treatment, prevention or delay of progression of said dyskinesia, wherein the therapy comprises the administration of levodopa. The present disclosure neuronal nicotinic receptor agonists selective for α7 subtype that are useful for improving dyskinesias associated with dopamine agonist therapy. Compounds, compositions containing such compounds, and methods of using such compound and compositions are described herein.

### BACKGROUND OF THE INVENTION

Many of the leading treatments for diseases lead to undesired side effects. For instance, levodopa, the standard for Parkinson's disease treatment, is associated with debilitating abnormal involuntary movements or dyskinesias. These motor abnormalities may occur after only a few months of treatment and affect the majority of patients within 5-10 years. They can be quite incapacitating and represent a major complication in Parkinson's disease management. Currently there are only limited therapeutic options for dyskinesias. Parkinson's disease is extremely common amongst those over 65, and age group that, in North America, is predicted to rise from 12% to 24% over the next 30 year. The overall prevalence of Parkinson's disease in this population is in the order of 1.5-2% and increases with age. Therefore, additional treatments are needed for this disabling complication of levodopa therapy.

Nicotinic acetylcholine receptors (nAChRs) are widely distributed throughout the central (CNS) and peripheral (PNS) nervous systems. Such receptors play an important role in regulating CNS function, particularly by modulating release of a wide range of neurotransmitters, including, but not necessarily limited to, acetylcholine, norepinephrine, dopamine, serotonin, and GABA. Consequently, nicotinic receptors mediate a very wide range of physiological effects, and have been targeted for therapeutic treatment of disorders relating to cognitive function, learning and memory, neurodegeneration, pain, inflammation, psychosis, sensory gating, mood, and emotion, among other conditions.

Many subtypes of the nAChR exist in the CNS and periphery. Each subtype has a different effect on regulating the overall physiological function. Typically, nAChRs are ion channels that are constructed from a pentameric assembly of subunit proteins. At least 12 subunit proteins, α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition (α4)2(β2)3 (the α4β2 subtype), while another major population of receptors is comprised of homomeric (α7)5 (the α7 subtype) receptors.

Certain compounds, like the plant alkaloid nicotine, interact with all subtypes of the nAChRs, accounting for the profound physiological effects of this compound. While nicotine has been demonstrated to have many beneficial properties, not all of the effects mediated by nicotine are desirable. For example, nicotine exerts gastrointestinal and cardiovascular side effects that interfere at therapeutic doses, and its addictive nature and acute toxicity are well-known. Ligands that select for interaction with only certain subtypes of the nAChR offer potential for achieving beneficial therapeutic effects with an improved margin for safety.

The α7 and α4β2 nAChRs have been shown to play a significant role in enhancing cognitive function, including aspects of learning, memory and attention (Levin, E. D., J. Neurobiol. 53: 633-640, 2002). For example, α7 nAChRs have been linked to conditions and disorders related to attention deficit disorder, attention deficit hyperactivity disorder (ADHD), schizophrenia, Alzheimer's disease (AD), mild cognitive impairment, senile dementia, dementia associated with Lewy bodies, dementia associated with Down's syndrome, AIDS dementia, and Pick's disease, as well as inflammation. The α4β2 receptor subtype is implicated in attention, cognition, epilepsy, and pain control (Paterson and Norberg, Progress in Neurobiology 61 75-111, 2000) as well as smoking cessation or nicotine withdrawal syndrome.

Several lines of evidence suggest that targeting α7 neuronal nicotinic receptors (NNRs) have the potential to result in cognitive and functional improvements in patients with schizophrenia. Abnormal α7 NNR activity in patients with schizophrenia is reported in brain areas central to cognitive processing. Patients with schizophrenia have decreased expression of α7 NNRs in the hippocampus and frontal cortex. The chromosomal site for the α7-nicotinic receptor subunit gene is a site of heritability for schizophrenia with polymorphisms associated with a deficit in P50 sensory gating. Smoking rates in patients with schizophrenia are greater than that in the general population; and it is hypothesized that smoking may represent an attempt to compensate for deficits in nicotinic receptor activity that are related to the abnormalities in cognition central to the disease.

The activity at both α7 and α42 nAChRs can be modified or regulated by the administration of subtype selective nAChR ligands. The ligands can exhibit antagonist, agonist, or partial agonist properties. Compounds that function as allosteric modulators are also known.

Although compounds that nonselectively demonstrate activity at a range of nicotinic receptor subtypes including α7 nAChRs are known, it would be beneficial to provide compounds that interact selectively with α7-containing neuronal nAChRs compared to other subtypes.

It would be beneficial to provide such nicotinic acetylcholine receptor ligand for improving symptoms associated with nAChR-mediated conditions, for example disorders such as schizophrenia and other related disorders. There remains a need for providing a neuronal nicotinic acetylcholine receptor agonist that treats such conditions in a safe and efficacious manner.

### SUMMARY OF THE INVENTION

The invention provides methods, compositions, and kits for the use of nicotinic receptor modulator. For example the methods, compositions, and kits described herein are used to reduce or eliminate a side effect. In some embodiments, the methods, compositions, and kits described herein are used to reduce or eliminate a side effect of a dopaminergic agent.

It has been found that α7 nicotinic acetylcholine receptor (nAChR) ligands, such as (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide or salts thereof, is effective for improving symptoms of cognitive deficits associated with schizophrenia in human adult nonsmoking patients. Moreover, administration of (4s)-4-(5-phenyl-1,3,4-thiadiazol-2-yloxy)-1-azatricyclo[3.3.1.1^{3,7}]decane to human patients reduced the severity of symptoms associated with schizophrenia disease in patients in a generally well tolerated manner. (4s)-4-(5-Phenyl-1,3,4-thiadiazol-2-yloxy)-1-azatricyclo[3.3.1.1^{3,7}]decane (Compound A, ABT-126), a neuronal nicotinic receptor agonist selective for α7 subtype of nicotinic acetylcholine receptors demonstrated beneficial dyskinesia-reducing effect in patients with side effects associated with dopamine agonist therapy. In particular, ABT-126, a neuronal nicotinic receptor agonist selective for α7 subtype of nicotinic acetylcholine receptors, demonstrates effect for reducing dyskinesias in patients with levodopa-induced dyskinesias.

A suitable medicament that is a neuronal nicotinic receptor agonist selective for α7 subtype is administered in sufficient doses to achieve effect in a patient. (4s)-4-(5-Phenyl-1,3,4-thiadiazol-2-yloxy)-1-azatricyclo[3.3.1.1^{3,7}]decane can be administered to a patient in need of treatment in doses of from about 6 mg to about 150 mg once daily (QD). Examples of suitable doses in the range of doses that can be administered are 10 mg QD, 25 mg QD, 50 mg QD, and 75 mg QD. The medicament is administered in a suitable fashion to achieve therapeutic effect. Once daily dosing for (4s)-4-(5-phenyl-1,3,4-thiadiazol-2-yloxy)-1-azatricyclo[3.3.1.1^{3,7}]decane is achieved via oral administration.

The compound can be administered to the patient in doses of from about 6 mg to about 150 mg once daily, and more particularly at 10 mg QD, 25 mg QD, 50 mg QD, or 75 mg QD.

A first aspect of the invention concerns the use of an α7-nAChR agonist or α7-nAChR positive allosteric modulator for the treatment (whether therapeutic or prophylactic), prevention or delay of progression of dyskinesia associated with dopamine agonist therapy in Parkinson's Disease.

One embodiment of said first aspect concerns the use of an α7-nAChR agonist for the treatment (whether therapeutic or prophylactic), prevention or delay of progression of dyskinesia associated with dopamine agonist therapy in Parkinson's Disease.

Another embodiment of said first aspect concerns the use of α7-nAChR positive allosteric modulator for the treatment (whether therapeutic or prophylactic), prevention or delay of progression of dyskinesia associated with dopamine agonist therapy in Parkinson's Disease.

A further aspect of the invention relates to a method for the treatment, prevention or delay of progression of dyskinesia associated with dopamine agonist therapy in Parkinson's Disease in a subject in need of such treatment, which comprises administering to said subject a therapeutically effective amount of an α7-nAChR agonist or an α7-nAChR positive allosteric modulator.

One embodiment of said further aspect relates to a method for the treatment, prevention or delay of progression of dyskinesia associated with dopamine agonist therapy in Parkinson's Disease in a subject in need of such treatment, which comprises administering to said subject a therapeutically effective amount of an α7-nAChR agonist.

Another embodiment of said further aspect relates to a method for the treatment, prevention or delay of progression of dyskinesia associated with dopamine agonist therapy in Parkinson's Disease in a subject in need of such treatment, which comprises administering to said subject a therapeutically effective amount of an α7-nAChR positive allosteric modulator.

A further aspect of the invention relates to a method for the treatment, prevention or delay of progression of dyskinesia associated with dopamine agonist therapy in Parkinson's Disease in a subject in need of such treatment, which comprises (i) diagnosing dyskinesia associated with dopamine agonist therapy in Parkinson's Disease in said subject and (ii) administering to said subject a therapeutically effective amount of an α7-nAChR agonist or an α7-nAChR positive allosteric modulator.

One embodiment of said further aspect relates to a method for the treatment, prevention or delay of progression of dyskinesia associated with dopamine agonist therapy in Parkinson's Disease in a subject in need of such treatment, which comprises (i) diagnosing dyskinesia associated with dopamine agonist therapy in Parkinson's Disease in said subject and (ii) administering to said subject a therapeutically effective amount of an α7-nAChR agonist. Another embodiment of said further aspect relates to a method for the treatment, prevention or delay of progression of dyskinesia associated with dopamine agonist therapy in Parkinson's Disease in a subject in need of such treatment, which comprises (i) diagnosing dyskinesia associated with dopamine agonist therapy in Parkinson's Disease in said subject and (ii) administering to said subject a therapeutically effective amount of an α7-nAChR positive allosteric modulator.

A further aspect of the invention relates to a pharmaceutical composition comprising an α7-nAChR agonist or an α7-nAChR positive allosteric modulator for the treatment, prevention or delay of progression of dyskinesia associated with dopamine agonist therapy in Parkinson's Disease.

One embodiment of said further aspect relates to a pharmaceutical composition comprising an α7-nAChR or an α7-nAChR positive allosteric modulator for the treatment, prevention or delay of progression of dyskinesia associated with dopamine agonist therapy in Parkinson's Disease.

Another embodiment of said further aspect relates to a pharmaceutical composition comprising an α7-nAChR or an α7-nAChR positive allosteric modulator for the treatment, prevention or delay of progression of dyskinesia associated with dopamine agonist therapy in Parkinson's Disease.

A further aspect of the invention relates to the use of an α7-nAChR agonist or an α7-nAChR positive allosteric modulator for the manufacture of a medicament for the treatment, prevention or delay of progression of dyskinesia associated with dopamine agonist therapy in Parkinson's Disease.

One embodiment of said further aspect relates to the use of an α7-nAChR a α7-nAChR agonist for the manufacture of a medicament for the treatment, prevention or delay of progression of dyskinesia associated with dopamine agonist therapy in Parkinson's Disease.

Another embodiment of said further aspect relates to the use of α7-nAChR positive allosteric modulator for the manufacture of a medicament for the treatment, prevention or delay of progression of dyskinesia associated with dopamine agonist therapy in Parkinson's Disease.

Additional aspects of the invention and further details are provided herein.

### BRIEF DESCRIPTION OF THE DRAWINGS

FIG. 1 graphically depicts decreases in levodopa-induced dyskinesias in MPTP-lesioned monkeys administered ABT-126.

FIG. 2 graphically depicts the reduced hourly time course of levodopa-induced dyskinesias in MPTP-lesioned monkeys administered ABT-126.

FIG. 3 graphically depicts reduced levodopa-induced dyskinesias with morning and afternoon treatment of levodopa.

### DETAILED DESCRIPTION OF THE INVENTION

All patents, patent applications, and literature references cited in the specification are herein incorporated by reference in their entirety.

For a variable that occurs more than one time in any substituent or in the compound of the invention or any other formulae herein, its definition on each occurrence is independent of its definition at every other occurrence. Combinations of substituents are permissible only if such combinations result in stable compounds. Stable compounds are compounds which can be isolated in a useful degree of purity from a reaction mixture.

As used throughout this specification and the appended claims, the following terms have the following meanings.

### ABBREVIATIONS

ANOVA, analysis of variance; LIDs, L-dopa-induced dyskinesias; MPTP, 1-methyl-4-phenyl-1,2,3,6-tetrahydropyridine; and nAChR, nicotinic acetylcholine receptor.

### Definition of Terms

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” means a divalent group derived from a straight or branched chain hydrocarbon of from 2 to 10 carbon atoms containing at least one double bond. Representative examples of alkenylene include, but are not limited to, —CH═CH—, —CH═CH₂CH₂—, and —CH═C(CH₃)CH₂—.

The term “alkenyloxy” as used herein, means an alkenyl group, as defined herein, appended to the parent molecular moiety through an oxygen atom. Representative examples of alkenyloxy include, but are not limited to, allyloxy, 2-butenyloxy and 3-butenyloxy.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkoxyalkyl” as used herein, means an alkoxyalkoxy group, as defined herein, appended to the parent molecular moiety through an alkyl group, as defined herein. Representative examples of alkoxyalkoxyalkyl include, but are not limited to, tert-butoxyethoxymethyl, ethoxymethoxymethyl, (2-methoxyethoxy)methyl, and 2-(2-methoxyethoxyl)ethyl.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylene” means a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl” as used herein, means an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonylalkyl include, but are not limited to, methylsulfonylmethyl and ethylsulfonylmethyl.

The term “alkylthio” as used herein, means an alkyl group, as defined herein, appended to the parent molecular moiety through a sulfur atom. Representative examples of alkylthio include, but are not limited to, methylthio, ethylthio, tert-butylthio, and hexylthio.

The term “alkylthioalkyl” as used herein, means an alkylthio group, as defined herein, appended to the parent molecular moiety through an alkyl group, as defined herein. Representative examples of alkylthioalkyl include, but are not limited to, methylthiomethyl and 2-(ethylthio)ethyl.

The term “alkynyl” as used herein, means a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “alkynylene” means a divalent group derived from a straight or branched chain hydrocarbon of from 2 to 10 carbon atoms containing at least one triple bond. Representative examples of alkynylene include, but are not limited to, —C≡C—, —CH₂C≡C—, —CH(CH₃)CH₂C≡C—, —C≡CCH₂—, and —C≡CCH(CH₃)CH₂—.

The term “alkynyloxy” as used herein, means an alkynyl group, as defined herein, appended to the parent molecular moiety through an oxygen atom. Representative examples of alkynyloxy include, but are not limited to, 2-propynyloxy and 2-butynyloxy.

The term “aryl,” as used herein, means phenyl, a bicyclic aryl or a tricyclic aryl. The bicyclic aryl is naphthyl, a phenyl fused to a cycloalkyl, or a phenyl fused to a cycloalkenyl. Representative examples of the bicyclic aryl include, but are not limited to, dihydroindenyl, indenyl, naphthyl, dihydronaphthalenyl, and tetrahydronaphthalenyl. The tricyclic aryl is anthracene or phenanthrene, or a bicyclic aryl fused to a cycloalkyl, or a bicyclic aryl fused to a cycloalkenyl, or a bicyclic aryl fused to a phenyl. Representative examples of tricyclic aryl ring include, but are not limited to, azulenyl, dihydroanthracenyl, fluorenyl, and tetrahydrophenanthrenyl.

The aryl groups of this invention can be substituted with 1, 2, 3, 4 or 5 substituents independently selected from alkenyl, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, carboxy, carboxyalkyl, cyano, cyanoalkyl, formyl, formylalkyl, halogen, haloalkyl, hydroxy, hydroxyalkyl, mercapto, nitro, —NZ₁Z₂, and (NZ₃Z₄)carbonyl.

The term “arylalkoxy” as used herein, means an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkoxycarbonyl” as used herein, means an arylalkoxy group, as defined herein, appended to the parent molecular moiety through a carbonyl group, as defined herein. Representative examples of arylalkoxycarbonyl include, but are not limited to, benzyloxycarbonyl and naphth-2-ylmethoxycarbonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkylthio” as used herein, means an arylalkyl group, as defined herein, appended to the parent molecular moiety through a sulfur atom. Representative examples of arylalkylthio include, but are not limited to, 2-phenylethylthio, 3-naphth-2-ylpropylthio, and 5-phenylpentylthio.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “aryloxy” as used herein, means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aryloxyalkyl” as used herein, means an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl and 3-bromophenoxymethyl.

The term “arylthio” as used herein, means an aryl group, as defined herein, appended to the parent molecular moiety through a sulfur atom. Representative examples of arylthio include, but are not limited to, phenylthio and 2-naphthylthio.

The term “arylthioalkyl” as used herein, means an arylthio group, as defined herein, appended to the parent molecular moiety through an alkyl group, as defined herein. Representative examples of arylthioalkyl include, but are not limited to, phenylthiomethyl, 2-naphth-2-ylthioethyl, and 5-phenylthiomethyl.

The term “AUC.” refers to the area under the plasma concentration time curve (AUC) extrapolated to infinity.

The term “azido” as used herein, means a —N₃ group.

The term “carbonyl” as used herein, means a —C(O)— group. The term “carboxy” as used herein, means a —CO₂H group. The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” as used herein, means a cyclic hydrocarbon containing from 3 to 8 carbons and containing at least one carbon-carbon double bond formed by the removal of two hydrogens. Representative examples of cycloalkenyl include, but are not limited to, 2-cyclohexen-1-yl, 3-cyclohexen-1-yl, 2,4-cyclohexadien-1-yl and 3-cyclopenten-1-yl.

The term “cycloalkyl” as used herein, means a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two adjacent or non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl groups of the invention are optionally substituted with 1, 2, 3, 4 or 5 substituents selected from the group consisting of alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkylthioalkyl, alkynyl, carboxy, cyano, formyl, haloalkoxy, haloalkyl, halogen, hydroxy, hydroxyalkyl, mercapto, oxo, —NZ₁Z₂, and (NZ₃Z₄)carbonyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “cycloalkylcarbonyl” as used herein, means cycloalkyl group, as defined herein, appended to the parent molecular moiety through a carbonyl group, as defined herein. Representative examples of cycloalkylcarbonyl include, but are not limited to, cyclopropylcarbonyl, 2-cyclobutylcarbonyl, and cyclohexylcarbonyl.

The term “cycloalkyloxy” as used herein, means cycloalkyl group, as defined herein, appended to the parent molecular moiety through an oxygen atom, as defined herein. Representative examples of cycloalkyloxy include, but are not limited to, cyclopropyloxy, cyclobutyloxy, cyclopentyloxy, cyclohexyloxy, cycloheptyloxy, and cyclooctyloxy.

The term “cycloalkylthio” as used herein, means cycloalkyl group, as defined herein, appended to the parent molecular moiety through a sulfur atom, as defined herein. Representative examples of cycloalkylthio include, but are not limited to, cyclopropylthio, cyclobutylthio, cyclopentylthio, cyclohexylthio, cycloheptylthio, and cyclooctylthio.

The term “ethylenedioxy” as used herein, means —O(CH₂)₂O— group wherein the oxygen atoms of the ethylenedioxy group are attached to the parent molecular moiety through one carbon atom forming a 5 membered ring or the oxygen atoms of the ethylenedioxy group are attached to the parent molecular moiety through two adjacent carbon atoms forming a six membered ring.

The term “formyl” as used herein, means a —C(O)H group.

The term “formylalkyl” as used herein, means a formyl group, as defined herein, appended to the parent molecular moiety through an alkyl group, as defined herein. Representative examples of formylalkyl include, but are not limited to, formylmethyl and 2-formylethyl.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic heteroaryl. The monocyclic heteroaryl is a 5 or 6 membered ring that contains at least one heteroatom selected from the group consisting of nitrogen, oxygen and sulfur. The 5 membered ring contains two double bonds and the 6 membered ring contains three double bonds. The 5 or 6 membered heteroaryl is connected to the parent molecular moiety through any carbon atom or any substitutable nitrogen atom contained within the heteroaryl, provided that proper valance is maintained. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any substitutable nitrogen atom contained within the bicyclic heteroaryl, provided that proper valance is maintained. Representative examples of bicyclic heteroaryl include, but are not limited to, azaindolyl, benzimidazolyl, benzofuranyl, benzoxadiazolyl, benzoisoxazole, benzoisothiazole, benzooxazole, 1,3-benzothiazolyl, benzothienyl(or benzothiophenyl), cinnolinyl, furopyridine, indolyl, indazolyl, indolinonyl, isobenzofuran, isoindolyl, isoquinolinyl, naphthyridinyl, oxadiazolyl, oxazolopyridine, quinolinyl, quinoxalinyl, thiadiazolyl, and thienopyridinyl.

The heteroaryl groups of the invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂ and (NZ₃Z₄)carbonyl. Heteroaryl groups of the invention that are substituted with a hydroxy group may be present as tautomers. The heteroaryl groups of the invention encompasses all tautomers including non-aromatic tautomers. In addition, the nitrogen heteroatoms can be optionally quaternized or oxidized to the N-oxide.

The term “heteroarylalkoxy” as used herein, means a heteroaryl group, as defined herein, appended to the parent molecular moiety through an alkoxy group, as defined herein. Representative examples of heteroarylalkoxy include, but are not limited to, fur-3-ylmethoxy, 1H-imidazol-2-ylmethoxy, 1H-imidazol-4-ylmethoxy, 1-(pyridin-4-yl)ethoxy, pyridin-3-ylmethoxy, 6-chloropyridin-3-ylmethoxy, pyridin-4-ylmethoxy, (6-(trifluoromethyl)pyridin-3-yl)methoxy, (6-(cyano)pyridin-3-yl)methoxy, (2-(cyano)pyridin-4-yl)methoxy, (5-(cyano)pyridin-2-yl)methoxy, (2-(chloro)pyridin-4-yl)methoxy, pyrimidin-5-ylmethoxy, 2-(pyrimidin-2-yl)propoxy, thien-2-ylmethoxy, and thien-3-ylmethoxy.

The term “heteroarylalkyl” as used herein, means a heteroaryl, as defined herein, appended to the parent molecular moiety through an alkyl group, as defined herein. Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl, and thien-3-ylmethyl.

The term “heteroarylalkylcarbonyl” as used herein, means a heteroarylalkyl, as defined herein, appended to the parent molecular moiety through a carbonyl group, as defined herein.

The term “heteroarylalkylthio” as used herein, means a heteroarylalkyl group, as defined herein, appended to the parent molecular moiety through a sulfur atom. Representative examples of heteroarylalkylthio include, but are not limited to, fur-3-ylmethylthio, 1H-imidazol-2-ylmethylthio, 1H-imidazol-4-ylmethylthio, pyridin-3-ylmethylthio, 6-chloropyridin-3-ylmethylthio, pyridin-4-ylmethylthio, (6-(trifluoromethyl)pyridin-3-yl)methylthio, (6-(cyano)pyridin-3-yl)methylthio, (2-(cyano)pyridin-4-yl)methylthio, (5-(cyano)pyridin-2-yl)methylthio, (2-(chloro)pyridin-4-yl)methylthio, pyrimidin-5-ylmethylthio, 2-(pyrimidin-2-yl)propylthio, thien-2-ylmethylthio, and thien-3-ylmethylthio.

The term “heteroarylcarbonyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl, and thien-3-ylcarbonyl.

The term “heteroaryloxy” as used herein, means a heteroaryl group, as defined herein, appended to the parent molecular moiety through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy, pyrimidin-5-yloxy, pyrimidin-2-yloxy, thien-2-yloxy, and thien-3-yloxy.

The term “heteroaryloxyalkyl” as used herein, means a heteroaryloxy group, as defined herein, appended to the parent molecular moiety through an alkyl group, as defined herein. Representative examples of heteroaryloxyalkyl include, but are not limited to, pyridin-3-yloxymethyl and 2-quinolin-3-yloxyethyl.

The term “heteroarylthio” as used herein, means a heteroaryl group, as defined herein, appended to the parent molecular moiety through a sulfur atom. Representative examples of heteroarylthio include, but are not limited to, pyridin-3-ylthio and quinolin-3-ylthio.

The term “heteroarylthioalkyl” as used herein, means a heteroarylthio group, as defined herein, appended to the parent molecular moiety through an alkyl group, as defined herein. Representative examples of heteroarylthioalkyl include, but are not limited to, pyridin-3-ylthiomethyl, and 2-quinolin-3-ylthioethyl.

The term “heterocycle” or “heterocyclic” as used herein, means a monocyclic heterocycle, a bicyclic heterocycle or a tricyclic heterocycle. The monocyclic heterocycle is a 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle.

Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a 5 or 6 membered monocyclic heterocycle fused to a phenyl group, or a 5 or 6 membered monocyclic heterocycle fused to a cycloalkyl, or a 5 or 6 membered monocyclic heterocycle fused to a cycloalkenyl, or a 5 or 6 membered monocyclic heterocycle fused to a monocyclic heterocycle. The bicyclic heterocycle is connected to the parent molecular moiety through any carbon atom or any nitrogen atom contained within the bicyclic heterocycle. Representative examples of bicyclic heterocycle include, but are not limited to, 1,3-benzodioxolyl, 1,3-benzodithiolyl, 2,3-dihydro-1,4-benzodioxinyl, benzodioxolyl, 2,3-dihydro-1-benzofuranyl, 2,3-dihydro-1-benzothienyl, chromenyl and 1,2,3,4-tetrahydroquinolinyl. The tricyclic heterocycle is a bicyclic heterocycle fused to a phenyl, or a bicyclic heterocycle fused to a cycloalkyl, or a bicyclic heterocycle fused to a cycloalkenyl, or a bicyclic heterocycle fused to a monocyclic heterocycle. The tricyclic heterocycle is connected to the parent molecular moiety through any carbon atom or any nitrogen atom contained within the tricyclic heterocycle. Representative examples of tricyclic heterocycle include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The heterocycles of this invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, oxo, —NZ₁Z₂ and (NZ₃Z₄)carbonyl.

The term “heterocyclealkoxy” as used herein, means a heterocycle group, as defined herein, appended to the parent molecular moiety through an alkoxy group, as defined herein. Representative examples of heterocyclealkoxy include, but are not limited to, 2-pyridin-3-ylethoxy, 3-quinolin-3-ylpropoxy, and 5-pyridin-4-ylpentyloxy.

The term “heterocyclealkyl” as used herein, means a heterocycle, as defined herein, appended to the parent molecular moiety through an alkyl group, as defined herein. Representative examples of heterocyclealkyl include, but are not limited to, piperidin-4-ylmethyl, piperazin-1-ylmethyl, 3-methyl-1-pyrrolidin-1-ylbutyl, (1R)-3-methyl-1-pyrrolidin-1-ylbutyl, (1S)-3-methyl-1-pyrrolidin-1-ylbutyl.

The term “heterocyclealkylcarbonyl” as used herein, means a heterocyclealkyl, as defined herein, appended to the parent molecular moiety through a carbonyl group, as defined herein.

Representative examples of heterocyclealkylcarbonyl include, but are not limited to, piperidin-4-ylmethylcarbonyl, piperazin-1-ylmethylcarbonyl, 3-methyl-1-pyrrolidin-1-ylbutylcarbonyl, (1R)-3-methyl-1-pyrrolidin-1-ylbutylcarbonyl, (1S)-3-methyl-1-pyrrolidin-1-ylbutylcarbonyl.

The term “heterocyclealkylthio” as used herein, means a heterocyclealkyl group, as defined herein, appended to the parent molecular moiety through a sulfur atom. Representative examples of heterocyclealkylthio include, but are not limited to, 2-pyridin-3-ylethythio, 3-quinolin-3-ylpropythio, and 5-pyridin-4-ylpentylthio.

The term “heterocyclecarbonyl” as used herein, means a heterocycle, as defined herein, appended to the parent molecular moiety through a carbonyl group, as defined herein. The term “heterocyclecarbonylalkyl” as used herein, means a heterocyclecarbonyl, as defined herein, appended to the parent molecular moiety through an alkyl group, as defined herein.

The term “heterocycleoxy” as used herein, means a heterocycle group, as defined herein, appended to the parent molecular moiety through an oxygen atom. Representative examples of heterocycleoxy include, but are not limited to, pyridin-3-yloxy and quinolin-3-yloxy.

The term “heterocycleoxyalkyl” as used herein, means a heterocycleoxy group, as defined herein, appended to the parent molecular moiety through an alkyl group, as defined herein. Representative examples of heterocycleoxyalkyl include, but are not limited to, pyridin-3-yloxymethyl and 2-quinolin-3-yloxyethyl.

The term “heterocyclethio” as used herein, means a heterocycle group, as defined herein, appended to the parent molecular moiety through a sulfur atom. Representative examples of heterocyclethio include, but are not limited to, pyridin-3-ylthio and quinolin-3-ylthio.

The term “heterocyclethioalkyl” as used herein, means a heterocyclethio group, as defined herein, appended to the parent molecular moiety through an alkyl group, as defined herein. Representative examples of heterocyclethioalkyl include, but are not limited to, pyridin-3-ylthiomethyl, and 2-quinolin-3-ylthioethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “hydroxy-protecting group” or “O-protecting group” means a substituent which protects hydroxy groups against undesirable reactions during synthetic procedures. Examples of hydroxy-protecting groups include, but are not limited to, substituted methyl ethers, for example, methoxymethyl, benzyloxymethyl, 2-methoxyethoxymethyl, 2-(trimethylsilyl)-ethoxymethyl, benzyl, and triphenylmethyl; tetrahydropyranyl ethers; substituted ethyl ethers, for example, 2,2,2-trichloroethyl and t-butyl; silyl ethers, for example, trimethylsilyl, t-butyldimethylsilyl and t-butyldiphenylsilyl; cyclic acetals and ketals, for example, methylene acetal, acetonide and benzylidene acetal; cyclic ortho esters, for example, methoxymethylene; cyclic carbonates; and cyclic boronates. Commonly used hydroxy-protecting groups are disclosed in T. W. Greene and P. G. M. Wuts, Protective Groups in Organic Synthesis, 3rd edition, John Wiley & Sons, New York (1999).

The term “lower alkenyl” as used herein, is a subset of alkenyl, as defined herein, and means an alkenyl group containing from 2 to 4 carbon atoms. Examples of lower alkenyl are ethenyl, propenyl, and butenyl.

The term “lower alkoxy” as used herein, is a subset of alkoxy, as defined herein, and means a lower alkyl group, as defined herein, appended to the parent molecular moiety through an oxygen atom, as defined herein. Representative examples of lower alkoxy include, but are not limited to, methoxy, ethoxy, propoxy, 2-propoxy, butoxy, and tert-butoxy.

The term “lower alkyl” as used herein, is a subset of alkyl as defined herein and means a straight or branched chain hydrocarbon group containing from 1 to 4 carbon atoms. Examples of lower alkyl are methyl, ethyl, n-propyl, iso-propyl, n-butyl, iso-butyl, sec-butyl, and tert-butyl.

The term “lower alkylthio” as used herein, is a subset of alkylthio, means a lower alkyl group, as defined herein, appended to the parent molecular moiety through a sulfur atom. Representative examples of lower alkylthio include, but are not limited to, methylthio, ethylthio, and tert-butylthio.

The term “lower alkynyl” as used herein, is a subset of alkynyl, as defined herein, and means an alkynyl group containing from 2 to 4 carbon atoms. Examples of lower alkynyl are ethynyl, propynyl, and butynyl.

The term “lower haloalkoxy” as used herein, is a subset of haloalkoxy, as defined herein, and means a straight or branched chain haloalkoxy group containing from 1 to 4 carbon atoms. Representative examples of lower haloalkoxy include, but are not limited to, trifluoromethoxy, trichloromethoxy, dichloromethoxy, fluoromethoxy, and pentafluoroethoxy.

The term “lower haloalkyl” as used herein, is a subset of haloalkyl, as defined herein, and means a straight or branched chain haloalkyl group containing from 1 to 4 carbon atoms. Representative examples of lower haloalkyl include, but are not limited to, trifluoromethyl, trichloromethyl, dichloromethyl, fluoromethyl, and pentafluoroethyl.

The term “mercapto” as used herein, means a —SH group.

The term “mercaptoalkyl” as used herein, means a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “methylenedioxy” as used herein, means a —OCH₂O— group wherein the oxygen atoms of the methylenedioxy are attached to the parent molecular moiety through two adjacent carbon atoms.

The term “nitrogen protecting group” as used herein, means those groups intended to protect an amino group against undesirable reactions during synthetic procedures. Preferred nitrogen protecting groups are acetyl, benzoyl, benzyl, benzyloxycarbonyl (Cbz), formyl, phenylsulfonyl, tert-butoxycarbonyl (Boc), tert-butylacetyl, trifluoroacetyl, and triphenylmethyl (trityl).

The term “nitro” as used herein, means a —NO₂ group.

The term “NZ₁Z₂” as used herein, means two groups, Z₁ and Z₂, which are appended to the parent molecular moiety through a nitrogen atom. Z₁ and Z₂ are each independently selected from the group consisting of hydrogen, alkyl, alkylcarbonyl, alkoxycarbonyl, aryl, arylalkyl, formyl and (NZ₅Z₆)carbonyl. In certain instances within the invention, Z₁ and Z₂ taken together with the nitrogen atom to which they are attached form a heterocyclic ring. Representative examples of NZ₁Z₂ include, but are not limited to, amino, methylamino, acetylamino, acetylmethylamino, phenylamino, benzylamino, azetidinyl, pyrrolidinyl and piperidinyl.

The term “NZ₃Z₄” as used herein, means two groups, Z₃ and Z₄, which are appended to the parent molecular moiety through a nitrogen atom. Z₃ and Z₄ are each independently selected from the group consisting of hydrogen, alkyl, aryl and arylalkyl. Representative examples of NZ₃Z₄ include, but are not limited to, amino, methylamino, phenylamino and benzylamino.

The term “NZ₅Z₆” as used herein, means two groups, Z₅ and Z₆, which are appended to the parent molecular moiety through a nitrogen atom. Z₅ and Z₆ are each independently selected from the group consisting of hydrogen, alkyl, aryl and arylalkyl. Representative examples of NZ₅Z₆ include, but are not limited to, amino, methylamino, phenylamino and benzylamino.

The term “(NZ₃Z₄)carbonyl” as used herein, means a NZ₃Z₄ group, as defined herein, appended to the parent molecular moiety through a carbonyl group, as defined herein. Representative examples of (NZ₃Z₄)carbonyl include, but are not limited to, aminocarbonyl, (methylamino)carbonyl, (dimethylamino)carbonyl, and (ethylmethylamino)carbonyl.

The term “oxo” as used herein, means a ═O moiety.

The term “sulfinyl” as used herein, means a —S(O)— group.

The term “sulfonyl” as used herein, means a —SO₂— group.

The term “tautomer” as used herein means a proton shift from one atom of a compound to another atom of the same compound wherein two or more structurally distinct compounds are in equilibrium with each other.

The term “pharmaceutically suitable excipient” refers to a solid, semi-solid or liquid fillers, diluents, encapsulating material, formulation auxiliary suitable for administering to a subject. Examples of pharmaceutically suitable excipients include, but are not limited to, sugars, cellulose and derivatives thereof, oils, glycols, solutions, buffers, colorants, releasing agents, coating agents, sweetening agents, flavoring agents, perfuming agents, and the like. Such therapeutic compositions may be administered parenterally, intracisternally, orally, rectally, intraperitoneally or by other dosage forms known in the art.

The term “therapeutically suitable metabolite” refers to a pharmaceutically active compound formed by the in vivo biotransformation of compounds of formula (I-V).

The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the compounds of formula (I-V) for example, by hydrolysis in blood.

The term “prodrug,” refers to compounds that contain, but are not limited to, substituents known as “therapeutically suitable esters.” The term “therapeutically suitable ester,” refers to alkoxycarbonyl groups appended to the parent molecule on an available carbon atom. More specifically, a “therapeutically suitable ester,” refers to alkoxycarbonyl groups appended to the parent molecule on one or more available aryl, cycloalkyl and/or heterocycle groups as defined herein. Compounds containing therapeutically suitable esters are an example, but are not intended to limit the scope of compounds considered to be prodrugs. Examples of prodrug ester groups include pivaloyloxymethyl, acetoxymethyl, phthalidyl, indanyl and methoxymethyl, as well as other such groups known in the art. Other examples of prodrug ester groups are found in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987.

“Dopamine agonist therapy” is generally used in the treatment of Parkinson's Disease. The term “dopamine agonist therapy” as used herein, unless indicated otherwise, means any therapy that increases dopamine receptor stimulation, including, but not limited to, therapies that directly stimulate dopamine receptors (such as administration of bromocriptine) and therapies that increase the levels of dopamine (such as administration of levodopa or of drugs which inhibit dopamine metabolism). Dopamine agonist therapies include, but are not limited to, therapies which comprise the administration of one or more of the following agents: levodopa (or L-dopa being a precursor of dopamine); levodopa in combination with a levodopa decarboxylase inhibitor, such as carbidopa or benserazide; levodopa in combination with a catechol-0-methyl transferase inhibitor, such as tolcapone or entacapone; a monoamine oxidase B-inhibitor, such as selegiline or rasagiline; a dopamine receptor agonist, such as bromocriptine, per-golide, pramipexole, ropinirole, cabergoline, apomorphine or lisuride.

The term “dopamine agonist” as used herein, unless otherwise indicated, means any agent that increases dopamine receptor stimulation. Preferred dopamine agonists are levodopa; levodopa in combination with a levodopa decarboxylase inhibitor, levodopa in combination with a catechol-0-methyl transferase inhibitor, a monoamine oxidase B-inhibitor and a dopamine receptor agonist.

In one embodiment of the invention, the therapy comprises the administration of levodopa. Due to prevalence of associated dyskinesia, the daily dosage of levodopa for an effective dopamine agonist therapy of Parkinson's Disease needs to be determined for each patient individually and ranges typically from 250 to 1500 mg. Said total daily dose is distributed between 2-6 administrations per day, e.g. 3-6 administrations of 50-100 mg per administration. Usually, the daily dosage of levodopa needed for an effective therapy increases during the course of the therapy.

In one embodiment of the invention, the therapy comprises the administration of levodopa in combination with a levodopa decarboxylase inhibitor, such as carbidopa or benserazide.

The term “dyskinesia associated with dopamine agonist therapy”, as used herein, unless otherwise indicated, means any dyskinesia which accompanies, or follows in the course of, dopamine agonist therapy, or which is caused by, related to, or exacerbated by dopamine agonist therapy, wherein dyskinesia and dopamine agonist therapy are as defined above. Such dyskinesia often, although not exclusively, occurs as a side-effect of said dopamine agonist therapies of Parkinson's Disease.

Characteristics of such dyskinesias include motor impairment, e.g. the appearance of slow and uncoordinated involuntary movements, shaking, stiffness and problems walking. For example, patients treated with levodopa often have reduced symptoms of Parkinson's disease but they experience increasing difficulties to remain standing or even sitting. After prolonged use of levodopa, a majority of patients develop such dyskinesia. Dyskinesia can occur at any time during the cycle of treatment with levodopa.

The terms “weight percent” or “percent by weight” or “% by weight” or “wt %” denote the weight of an individual component in a composition or mixture as a percentage of the weight of the composition or mixture.

Substituents attached to a cyclic moiety, for instance a cycloalkyl, aryl, or heterocycloalkyl moiety, can be represented as not bound to any particular atom, but rather as attached to bonds that perpendicularly intersect a side of the cyclic group. This notation is meant to indicate that the substituent can be bound to one of two or more atoms of the cyclic group.

Although typically it may be recognized that an asterisk is used to indicate that the exact subunit composition of a receptor is uncertain, for example a3b4* indicates a receptor that contains the a3 and b4 proteins in combination with other subunits, the term α7 as used herein is intended to include receptors wherein the exact subunit composition is both certain and uncertain. For example, as used herein α7 includes homomeric (α7)5 receptors and α7* receptors, which denote a nAChR containing at least one α7 subunit.

### Compounds of the Invention

Compounds which may be used in the methods and compositions of the invention are those of the Formula (I),

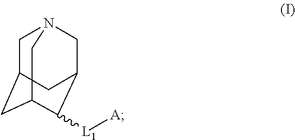

or a pharmaceutically acceptable salt or prodrug thereof, wherein

L₁ is —O— or —NRₐ—;

A is —Ar₁, —Ar₂-L₂-Ar₃ or —Ar₄-L₃-Ar₅;

Ar₁ is aryl or heteroaryl;

Ar₂ is aryl or monocyclic heteroaryl;

Ar₃ is aryl or heteroaryl;

Ar₄ is a bicyclic heteroaryl;

Ar₅ is aryl or heteroaryl;

L₂ is a bond, —O—, —NRₐ—, —CH₂—, or —C(O)NRₐ—;

L₃ is a bond, —O—, —NRₐ— or —CH₂—; and

Rₐ is hydrogen or alkyl.

Another embodiment is a compound of formula (II),

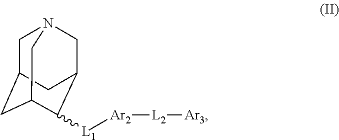

or a therapeutically suitable salt or prodrug thereof, wherein

Ar₂ is selected from

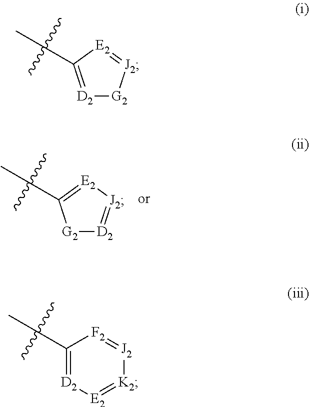

D₂, E₂, F₂, J₂, and K₂ are each independently —CT₂ or N;

G₂ is O, —NR₂ₐ, or S;
in each group of (i), (ii), and (iii), one substituent represented by T₂, or R₂ₐ wherein R₂ₐ is T₂, is -L₂-Ar₃ and the other substituents represented by T₂ are hydrogen, alkyl, alkoxy, alkoxycarbonyl, cyano, halo, nitro, or —NR_{b}R_{c};
R₂ₐ is hydrogen, alkyl, or T₂; and
R_{b} and R_{c} are each independently hydrogen, alkyl, alkoxycarbonyl or alkylcarbonyl.

Ar₃ is a group selected from

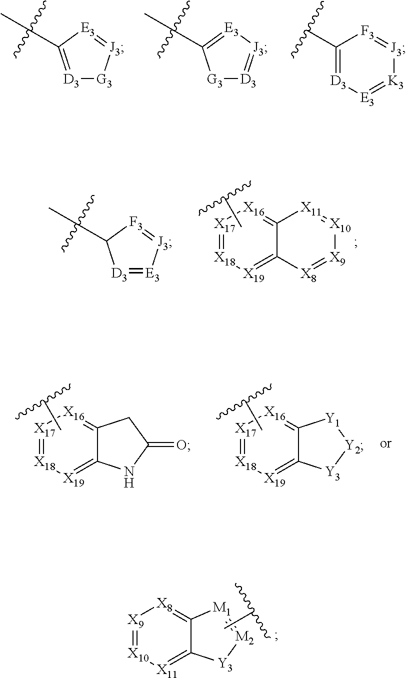

wherein D₃, E₃, F₃, J₃, K₃, X₈, X₉, X₁₀, and X₁₁ are each independently —CR₃ or N;
X₁₆, X₁₇, X₁₈, X₁₉, M₁, and M₂ are each independently —CR₃, N, or C;
G₃ is O, —NR₃ₐ, or S;
Y₁ is —CR₃ or N;
Y₂ is —CR₃ or N;
Y₃ is NH, O, or S;
R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano;
R₃ₐ is hydrogen, alkyl, alkylcarbonyl, tritylaryl, wherein aryl is preferably phenyl;
Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl;
one of X₁₆, X₁₇, X₁₈, and X₁₉, is C;
M₁ or M₂ is C;
L₁ is —O— or —NRₐ—;
L₂ is a bond, —O—, —NRₐ—, —CH₂—, or —C(O)NRₐ—; and
Rₐ is hydrogen or alkyl.

Another embodiment is a compound of formula (III),

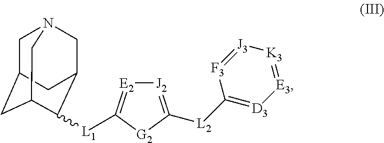

or a therapeutically suitable salt or prodrug thereof, wherein
E₂ and J₂ are each independently —CT₂ or N;
G₂ is O, —NR₂ₐ, or S;
T₂, at each occurrence, is independently hydrogen, alkyl, alkoxy, alkoxycarbonyl, cyano, halo, nitro, or —NR_{b}R_{c};
R₂ₐ is hydrogen, alkyl, or T₂;
R_{b} and R_{c} are each independently hydrogen, alkyl, alkoxycarbonyl or alkylcarbonyl;
D₃, E₃, F₃, J₃, and K₃ are each independently —CR₃ or N;
R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano;
Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl;
L₁ is —O— or —NRₐ—;
L₂ is a bond, —O—, —NRₐ—, —CH₂—, or —C(O)NRₐ—; and
Rₐ is hydrogen or alkyl.

Another embodiment is a compound of formula (IV),

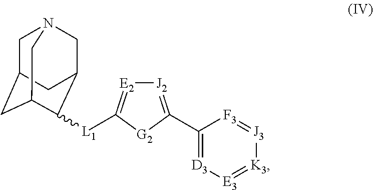

or a therapeutically suitable salt or prodrug thereof, wherein
E₂ and J₂ are each independently —CT₂ or N;
G₂ is O, —NR₂ₐ, or S;
T₂, at each occurrence, is independently hydrogen, alkyl, alkoxy, alkoxycarbonyl, cyano, halo, nitro, or —NR_{b}R_{c};
R₂ₐ is hydrogen, alkyl, or T₂;
R_{b} and R_{c} are each independently hydrogen, alkyl, alkoxycarbonyl or alkylcarbonyl;
D₃, E₃, F₃, J₃, and K₃ are each independently —CR₃ or N;
R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano; and
Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl.

Another embodiment is a compound of formula (V),

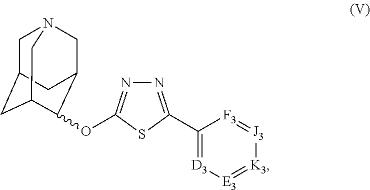

or a therapeutically suitable salt or prodrug thereof, wherein
D₃, E₃, F₃, J₃, and K₃ are each independently —CR₃ or N;
R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano; and
Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl.

Another embodiment is (4s)-4-(5-phenyl-1,3,4-thiadiazol-2-yloxy)-1-azatricyclo[3.3.1.1^{3,7}]decane (ABT-126 or Compound A).

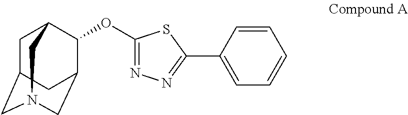 [SMILES: c1ccc(-c2nnc(O[C@@H]3C4CC5C[C@H]3C[N@](C5)C4)s2)cc1]

Alternatively, Compound A may also be called (1R,4R,5S)-4-(5-phenyl-1,3,4-thiadiazol-2-yloxy)-1-azatricyclo[3.3.1.1^{3,7}]decane. The preparation of compounds of the invention is disclosed in US Patent Application Publication No. 20080167336.

The nAChR ligand agonist may be a compound of the Formula (VI),

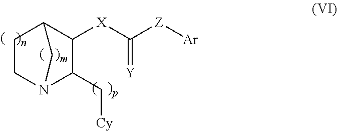

wherein
m is 2;
n is 1;
p is 1, 2, 3 or 4;
X is oxygen or NR′;
Y is oxygen or sulfur;
Z is NR′, a covalent bond or a linker species A;
A is selected from the group —CR′R″—, —CR′R″—CR′R″—, —CR′═CR′— and —C═C—;
when Z is a covalent bond or A, X must be nitrogen;
Ar is an unsubstituted or substituted, carbocyclic or heterocyclic, monocyclic or fused polycyclic aryl group;
Cy is an unsubstituted or substituted 5- or 6-membered heteroaromatic ring; and substituents are selected from the group consisting of alkyl, alkenyl, heterocyclyl, cycloalkyl, aryl, substituted aryl, arylalkyl, substituted arylalkyl, halo, —OR′, —NR′R″, —CF3, —CN, —NO2, —R′, —SR′, —N3, —C(═O)NR′R″, —NR′C(═O)R″, —C(═O)R′, —C(═O)OR′, —OC(═O)R′, —O(CR′R″)rC(═O)R′, —O(CR′R″)rNR″C(═O)R′, —O(CR′R″)—NR″SO₂R′, —OC(═O)NR′R″, —NR′C(═O)OR″, —SO2 R′, —SO₂NR′R″, and —NR′SO₂ R″;
wherein each of R′ and R″ individually is hydrogen, C1-C8 alkyl, C3-C8 cycloalkyl, heterocyclyl, aryl, or arylalkyl; or R′ and R″ can combine to form a 3 to 8 membered ring; and r is 1, 2, 3, 4, 5, or 6, or a pharmaceutically acceptable salt thereof.

Another compound which may be used for the methods may be TC-5619 (N-[2-(pyridin-3-ylmethyl)-1-azabicyclo[2.2.2]oct-3-yl]-1-benzofuran-2-carboxamide), which has been disclosed to be a neuronal nicotinic receptor agonist selective for α7 subtype

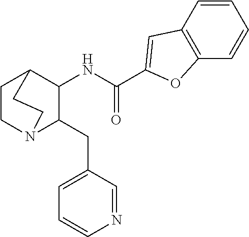 [SMILES: O=C(NC1C2CCN(CC2)C1Cc1cccnc1)c1cc2ccccc2o1]

The preparation of TC-5619 (N-[2-(pyridin-3-ylmethyl)-1-azabicyclo[2.2.2]oct-3-yl]-1-benzofuran-2-carboxamide) is disclosed U.S. Pat. No. 6,953,855.

The nAChR ligand agonist may be a compound of the Formula (VII),

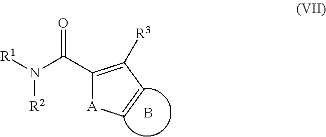

wherein
R1 represents 1-azabicyclo[2.2.2]oct-3-yl,
R2 represents hydrogen or C1-C6-alkyl,
R3 represents hydrogen, halohalogen or C1-C6-alkyl,
A represents oxygen or sulfur, and
the ring B represents benzo, pyrimido, pyrimidazo or pyridazino which is substituted by a radical selected from the group consisting of halogen, C₁-C₆-alkanoyl, carbamoyl, cyano, trifluoromethyl, trifluoromethoxy, nitro, amino, C₁-C₆-acylamino, C₁-C₆-alkyl, C₁-C₆-alkyoxy, C₁-C₆-alkylthio, C₁-C₆-alkylamino, heteroarylcarbonylamino, arylcarbonylamino, C₁-C₆-alkylsulfonyl-amino, di(C₁-C₄-alkylsulfonyl)amino, arylsulfonylamino, di(arylsulfonyl)amino, C₃-C₆-cycloalkylcarbonylmethyl, 1,3-dioxa-propane-1,3-diyl, amino(hydroxyimino)methyl and benzo, or a salt, a hydrate or a hydrate of a salt thereof.

The nAChR ligand agonist may be a compound of the Formula (VIII),

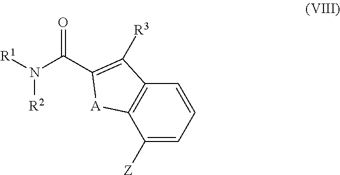

R1 represents 1-azabicyclo[2.2.2]oct-3-yl,
R2 represents hydrogen or C1-C6-alkyl,
R3 represents hydrogen, halogen or C1-C6-alkyl,
A represents oxygen or sulfur,
and Z represents halogen, formyl, carbamoyl, cyano, trifluoromethyl, trifluoromethoxy, nitro, amino, formamido, acetamido, C1-C6-alkyl, C1-C6-alkyoxy, C1-C6-alkylthio, C1-C6-alkylamino, heteroaryl-carbonylamino, arylcarbonylamino, C1-C4-alkylsulfonylamino, di(arylsulfonyl) amino, C3-C6-cycloalkylcarbonylmethyl or amino(hydroxyimino)methyl, or a salt, a hydrate or a hydrate of a salt thereof.

Another compound which may be used for the methods may be EVP-6124, which has been disclosed to be a neuronal nicotinic receptor partial agonist selective for α7 subtype. The preparation of EVP-6124 (N-[(3R)-1-azabicyclo[2.2.2]oct-3-yl]-7-chloro-1-benzothiophene-2-carboxamide) is disclosed in U.S. Pat. No. 7,732,477.

The nAChR ligand agonist may be (R)-7-chloro-N-(quinuclidin-3-yl)benzo[b]thiophene-2-carboxamide and has the following structure:

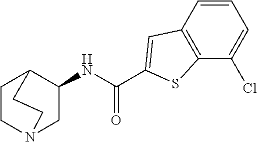 [SMILES: O=C(N[C@H]1CN2CCC1CC2)c1cc2cccc(Cl)c2s1]

### Salts of the Invention

The present compounds may exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts may be prepared during the final isolation and purification of the compounds or separately by reacting an amino group of the compounds with a suitable acid. For example, a compound may be dissolved in a suitable solvent, such as but not limited to methanol and water, and treated with at least one equivalent of an acid, like hydrochloric acid. The resulting salt may precipitate out and be isolated by filtration and dried under reduced pressure. Alternatively, the solvent and excess acid may be removed under reduced pressure to provide the salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, form ate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds may also be quaternized with alkyl chlorides, bromides, and iodides such as methyl, ethyl, propyl, isopropyl, butyl, lauryl, myristyl, stearyl, and the like.

Substantially pure crystalline salts of (4s)-4-(5-phenyl-1,3,4-thiadiazol-2-yloxy)-1-azatricyclo[3.3.1.1^{3,7}]decane are, for example, (4s)-4-(5-phenyl-1,3,4-thiadiazol-2-yloxy)-1-azatricyclo[3.3.1.1^{3,7}]decane L-bitartrate anhydrate, (4s)-4-(5-phenyl-1,3,4-thiadiazol-2-yloxy)-1-azatricyclo[3.3.1.1^{3,7}]decane L-bitartrate hydrate, (4s)-4-(5-phenyl-1,3,4-thiadiazol-2-yloxy)-1-azatricyclo[3.3.1.1^{3,7}]decane dihydrogen phosphate anhydrate, (4s-4-(5-phenyl-1,3,4-thiadiazol-2-yloxy)-1-azatricyclo[3.3.1.1^{3,7}]decane dihydrogen phosphate hydrate, (4s)-4-(5-phenyl-1,3,4-thiadiazol-2-yloxy)-1-azatricyclo[3.3.1.1^{3,7}]decane bisuccinate anhydrate, (4s)-4-(5-phenyl-1,3,4-thiadiazol-2-yloxy)-1-azatricyclo[3.3.1.1^{3,7}]decane bisuccinate hydrate, (4s)-4-(5-phenyl-1,3,4-thiadiazol-2-yloxy)-1-azatricyclo[3.3.1.1^{3,7}]decane hydrochloride quarterhydrate, (4s)-4-(5-phenyl-1,3,4-thiadiazol-2-yloxy)-1-azatricyclo[3.3.1.1^{3,7}]decane hydrochloride sesquihydrate, (4s)-4-(5-phenyl-1,3,4-thiadiazol-2-yloxy)-1-azatricyclo[3.3.1.1^{3,7}]decane dihydrogen citrate, (4s)-4-(5-phenyl-1,3,4-thiadiazol-2-yloxy)-1-azatricyclo[3.3.1.1^{3,7}]decane monohydrogen citrate, or (4s)-4-(5-phenyl-1,3,4-thiadiazol-2-yloxy)-1-azatricyclo[3.3.1.1^{3,7}]decane.

Basic addition salts may be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

### Amides, Esters and Prodrues of the Invention

Prodrugs are derivatives of an active drug designed to ameliorate some identified, undesirable physical or biological property. The physical properties are usually solubility (too much or not enough lipid or aqueous solubility) or stability related, while problematic biological properties include too rapid metabolism or poor bioavailability which itself may be related to a physicochemical property.

Prodrugs are usually prepared by: a) formation of ester, hemi esters, carbonate esters, nitrate esters, amides, hydroxamic acids, carbamates, imines, Mannich bases, and enamines of the active drug, b) functionalizing the drug with azo, glycoside, peptide, and ether functional groups, c) use of polymers, salts, complexes, phosphoramides, acetals, hemiacetals, and ketal forms of the drug. For example, see Andrejus Korolkovas's, “Essentials of Medicinal Chemistry”, John Wiley-Interscience Publications, John Wiley and Sons, New York (1988), pp. 97-118, which is incorporated in its entirety by reference herein.

Esters can be prepared from substrates of formula (I) containing either a hydroxyl group or a carboxy group by general methods known to persons skilled in the art. The typical reactions of these compounds are substitutions replacing one of the heteroatoms by another atom, for example:

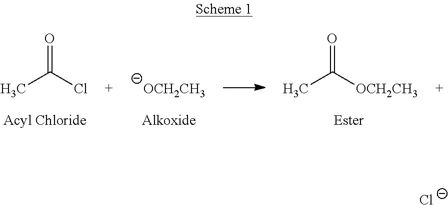

Amides can be prepared from substrates of formula (I) containing either an amino group or a carboxy group in similar fashion. Esters can also react with amines or ammonia to form amides.

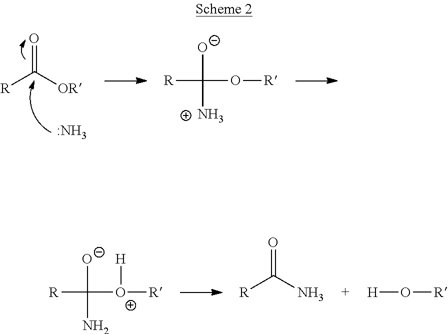

Another way to make amides from compounds of formula (I) is to heat carboxylic acids and amines together.

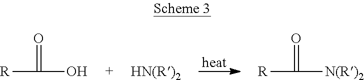

In Schemes 2 and 3, R and R′ are independently substrates of formulas I-V, alkyl or hydrogen.

### Optical Isomers-Diastereomers-Geometric Isomers

Asymmetric centers may exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described herein below and resolved by techniques well known in the art.

Geometric isomers may exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration. Furthermore, the invention contemplates the various isomers and mixtures thereof resulting from the disposal of substituents around an adamantane ring system. Two substituents around a single ring within an adamantane ring system are designated as being of Z or E relative configuration. For examples, see C. D. Jones, M. Kaselj, R. N. Salvatore, W. J. le Noble J. Org. Chem. 63: 2758-2760, 1998.

Compounds of the invention may exist as stereoisomers wherein, asymmetric or chiral centers are present. These stereoisomers are “R” or “S” depending on the configuration of substituents around the chiral element. The terms “R” and “S” used herein are configurations as defined in IUPAC 1974 Recommendations for Section E, Fundamental Stereochemistry, Pure Appl. Chem., 1976, 45: 13-30. The invention contemplates various stereoisomers and mixtures thereof and are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

More particularly, the compounds of the invention can exist in the forms represented by formula (Ia) and (Ib).

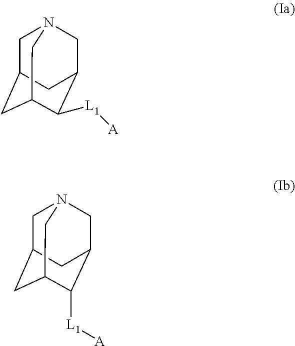

The aza-adamantane portion of isomer (Ia) and isomer (Ib) is not chiral, however the C-4 carbon at which L₁ is attached is considered pseudoasymmetric. Compounds represented by formula (Ia) and (Ib) are diastereomers. The configurational assignment of structures of formula (Ia) are assigned 4r in accordance with that described in Synthesis, 1992, 1080, Becker, D. P.; Flynn, D. L. and as defined in Stereochemistry of Organic Compounds, E. L. Eliel, S. H Wilen; John Wiley and Sons, Inc. 1994. In addition the configurational assignment of structures of formula (Ib) are assigned 4s using the same methods.

The isomers (Ia) and (Ib) may be synthesized separately using the individual steroisomers according to the Schemes or the Experimentals described herein. Alternatively, isomers (Ia) and (Ib) may be synthesized together after which the individual isomers may be separated by chromatographic methods from the mixture of both isomers when mixtures of stereoisomers are used in the synthesis. The mixtures of isomers may also be separated through fractional crystallization of salts of amines contained in the compounds of formula (I) made with enantiomerically pure carboxylic acids.

It is contemplated that a mixture of both isomers may be used to modulate the effects of nAChRs. Furthermore, it is contemplated that the individual isomers of formula (Ia) and (Ib) may be used alone to modulate the effects of nAChRs. Therefore, it is contemplated that either a mixture of the compounds of formula (Ia) and (Ib) or the individual isomers alone represented by the compounds of formula (Ia) or (Ib) would be effective in modulating the effects of nAChRs, and more particularly α7 nAChRs, α4β2 nAChRs, or a combination of α7nAChRs and α4β2 nAChRs and is thus within the scope of the invention.

More specifically, compounds contemplated as part of the invention include

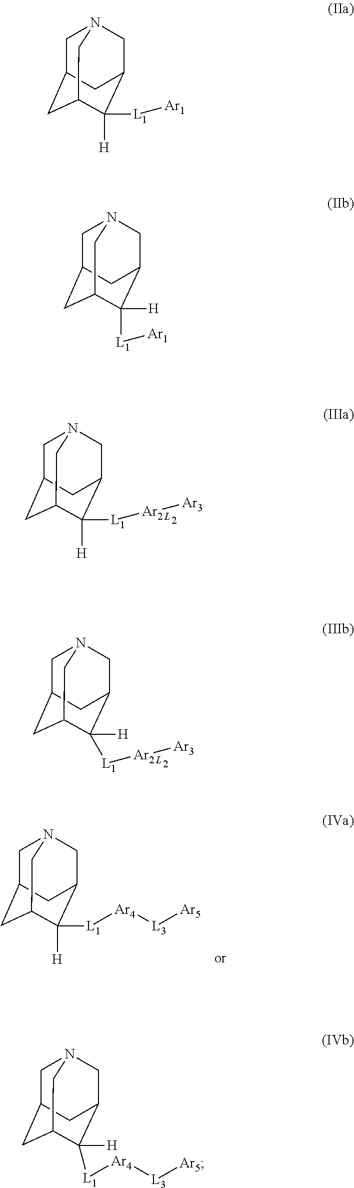

wherein L₁, L₂, L₃, Ar₁, Ar₂, Ar₃, Ar₄, and Ar₅ are defined herein.

### Isotone Enriched or Labeled Compounds

Compounds of the invention can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to, ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

In another embodiment, the isotope-labeled compounds contain deuterium (²H)tritium (³H) or ¹⁴C isotopes. Isotope-labeled compounds of this invention can be prepared by the general methods well known to persons having ordinary skill in the art. Such isotope-labeled compounds can be conveniently prepared by carrying out the procedures disclosed in the Examples disclosed herein and Schemes by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuteric acid such as D₂SO₄/D₂O In addition to the above, relevant procedures and intermediates are disclosed, for instance, in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO1995007271, WO2006008754; U.S. Pat. Nos. 7,538,189; 7,534,814; 7531685; 7528131; 7521421; 7514068; 7511013; and US Patent Application Publication Nos. 20090137457; 20090131485; 20090131363; 20090118238; 20090111840; 20090105338; 20090105307; 20090105147; 20090093422; 20090088416; and 20090082471, the methods are hereby incorporated by reference.

The isotope-labeled compounds of the invention may be used as standards to determine the effectiveness of nAChR ligands in binding assays. Isotope containing compounds have been used in pharmaceutical research to investigate the in vivo metabolic fate of the compounds by evaluation of the mechanism of action and metabolic pathway of the nonisotope-labeled parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

In addition, non-radio active isotope containing drugs, such as deuterated drugs called “heavy drugs,” can be used for the treatment of diseases and conditions related to nAChR activity. Increasing the amount of an isotope present in a compound above its natural abundance is called enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %.

Replacement of up to about 15% of normal atom with a heavy isotope has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci 1960 84: 736; Czakja D M et al., Am. J. Physiol. 1961 201: 357). Acute replacement of as high as 15%-23% in human fluids with deuterium was found not to cause toxicity (Blagojevic N et al. in “Dosimetry & Treatment Planning for Neutron Capture Therapy”, Zamenhof R, Solares G and Harling O Eds. 1994. Advanced Medical Publishing, Madison Wis. pp. 125-134; Diabetes Metab. 23: 251 (1997)).

Stable isotope labeling of a drug can alter its physico-chemical properties such as pKa and lipid solubility. These effects and alterations can affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. Accordingly, the incorporation of an isotope at a site of metabolism or enzymatic transformation will slow said reactions potentially altering the pharmacokinetic profile or efficacy relative to the non-istopic compound.

### Compositions of the Invention

Therapeutic compositions of the disclosure comprise an effective amount of an nAChR ligands of formulas I-V, or pharmaceutically acceptable salts, prodrugs, esters, amides or metabolites thereof formulated with one or more therapeutically suitable excipients.

In one embodiment, the therapeutically effective amount comprises an amount of the nAChR ligand from about 6 mg to about 150 mg. In another embodiment the therapeutically effective amount is selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 mg to about 50 mg, about 25 mg to about 50 mg, or about 50 mg to about 75 mg.

In another embodiment, the therapeutically effective amount of Compound A comprises an amount of the nAChR ligand from about 10 mg to about 150 mg. In another embodiment the therapeutically effective amount is selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 mg to about 50 mg, about 25 mg to about 50 mg, or about 50 mg to about 75 mg.

In another embodiment, the therapeutically effective amount of Compound A comprises an amount of the nAChR ligand from about 25 mg to about 75 mg. Compound A is administered in doses of 10 mg, 25 mg, 50 mg, or 75 mg.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar, buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution.

In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances.

Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N. Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The compounds of the invention can be used in the form of pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts can be prepared using various suitable acids for example, including, but are not limited to, acetic, adipic, alginic, citric, aspartic, benzoic, benzenesulfonic, butyric, camphoric, camphorsulfonic, carbonic, digluconic, glycerophosphoric, heptanoic, hexanoic, fumaric, hydrochloric, hydrobromic, hydroiodic, 2-hydroxyethansulfonic (isethionic), lactic, maleic, methanesulfonic, nicotinic, 2-naphthalenesulfonic, oxalic, pamoic, pectinic, persulfuric, 3-phenylpropionic, picric, pivalic, propionic, succinic, sulfuric, tartaric, thiocyanic, phosphoric, glutamatic, p-toluenesulfonic, and undecanoic acids.

Particular examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, tartaric acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A. C. S. Symposium Series, and in Edward B. Roche, ed., Bioreversible Carriers in Drug Design, American Pharmaceutical Association and Pergamon Press (1987).

The invention also contemplates pharmaceutically acceptable compounds that when administered to a patient in need may be converted through in vivo biotransformation into compounds of formula (I).

### Methods of the Present Invention

The compounds or compositions are administered to a patient in need of therapy or treatment for dyskinesias associated with dopamine agonist therapy, particularly levodopa. Any therapeutically effective neuronal nicotinic acetylcholine receptor agonist selective for α7subtype can be administered to patients who are receiving dopamine agonist therapy. Use in patients who have not yet received dopamine agonist therapy or no longer receive dopamine agonist therapy are contemplated.

The terms “subject” and “patient” are used interchangeably irrespective of whether the subject has or is currently undergoing any form of treatment.

Patients are administered a therapeutically effective amount of a suitable compound or composition. In one embodiment, the therapeutically effective amount comprises an amount of the nAChR ligand from about 6 mg to about 150 mg. In another embodiment the therapeutically effective amount is selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 mg to about 50 mg, about 25 mg to about 50 mg, or about 50 mg to about 75 mg.

In another embodiment, the therapeutically effective amount of Compound A comprises an amount of the nAChR ligand from about 10 mg to about 150 mg. In another embodiment the therapeutically effective amount is selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 mg to about 50 mg, about 25 mg to about 50 mg, or about 50 mg to about 75 mg.

In another embodiment, the therapeutically effective amount of Compound A comprises an amount of the nAChR ligand from about 25 mg to about 75 mg. Compound A is administered in doses of 10 mg, 25 mg, 50 mg, or 75 mg.

The compound or composition is delivered in a manner suitable for allowing the nAChR ligand to achieve therapeutic effect by interacting with the target receptor. The amount of the active agent administered can vary with the patient, the route of administration, and the result sought.

Optimum dosing regimens for particular patients can be determined by one skilled in the art using the guidance and dosing information provided herein.

In accordance with the present invention, the active agent can be administered in any convenient manner. Examples of suitable methods for administration include, but are not limited, orally, sublingually, rectally, parentally, (including subcutaneously intrathecally, intramuscularly, and intravenously), or transdermally. The most preferred route of administration is the oral route.

The active agents of the invention can be administered in the form of a pharmaceutical composition or compositions that contain one or both active agents in an admixture with a pharmaceutical carrier. The pharmaceutical composition can be in dosage unit form such as tablet, capsule, sprinkle capsule, granule, powder, syrup, suppository, injection, or the like.

Certain aspects of the invention are described in greater detail in the non-limiting Examples that follow:

### EXAMPLE 1

### Summary

The objective of the current study was to test the effect of the α7 nAChR agonist ABT-126 for its ability to reduce L-dopa-induced dyskinesias (LIDs) in MPTP-lesioned monkeys. All monkeys had been administered MPTP and exhibited mild to moderate parkinsonism. All monkeys were orally gavaged with L-dopa/carbidopa twice daily, which led to the development of stable abnormal involuntary movements or dyskinesias. The treatment groups were as follows: vehicle-treated (n=6), nicotine-treated (n=5), ABT-126-treated (set 1, n=5 and ABT-126-treated (set 2, n=5). These latter two sets of monkeys had previously been given ABT-894 and ABT-107 but using somewhat different treatment regimens. The present study was done after a 7 wk washout period, when LIDs were similar in all groups.

ABT-126 was administered orally in a small cracker 30 min before L-dopa (10 mg/kg) and carbidopa (2.5 mg/kg). Nicotine, included as a positive control, was provided in the drinking water. ABT-126 was tested at 0.03, 0.10, 0.30 and 1.0 mg/kg, with each dose of ABT-126 tested for 1 or 2 wk. The 0.03 mg/kg dose ABT-126 had no significant effect on LIDs. ABT-126 at 0.10 mg/kg reduced LIDs by ˜40%, while the 0.30 and 1.0 mg/kg ABT-126 doses decreased LIDs up to 60%. We also tested the effect of ABT-126 (1.0 mg/kg) for its ability to reduce LIDs at a higher dose of L-dopa (15 mg/kg)/carbidopa (3.75 mg/kg). The higher dose of L-dopa led to greater LID scores in vehicle treated monkeys. ABT-126 treatment reduced LIDs by 70% after the morning dose of L-dopa and 60% after the afternoon dose. The effect of ABT-126 was most pronounced during the latter weeks, possibly because of a greater decline with continued drug treatment. A washout is in progress to determine if LIDs return to vehicle-treated levels after ABT-126 discontinuation. There was no effect of the drug on parkinsonism or cognitive ability.

Overall, these data indicate that ABT-126 would be useful as an antidyskinetic drug in Parkinson's disease.

### EXAMPLE 1

### Experimental Design

Animals.

Adult male and female squirrel monkeys (Saimiri sciureus) (n=21) weighing 0.6-1.2 kg were obtained from World Wide Primates (Miami, Fla.). The monkeys were quarantined for one month, as mandated by California state regulations. All studies were performed according to the NIH Guide for the Care and Use of Laboratory Animals, and were approved by the Institutional Animal Care and Use Committee at SRI. Animals were housed separately in a room maintained at 27±3° C., with a 12:12-h light/dark cycle. Monkey chow, fruits, and vegetables were provided over the course of the day, with water freely available.

MPTP Lesions and Parkinsonian Ratings.

After quarantine, all monkeys were trained to perform various motor and other tasks for future assessment of parkinsonism and other behaviors (Quik et al. 2013b, Zhang et al. 2013, Quik et al. 2007, Quik et al. 2013a). They were then injected with saline (n=5) or 2.0 mg/kg 1-methyl-4-phenyl-1,2,3,6-tetrahydropyridine sc (MPTP; Sigma-Aldrich, St. Louis, Mo.) dissolved in saline (n=21), as described (Quik et al. 2013b, Zhang et al. 2013, Quik et al. 2007, Quik et al. 2013a). Parkinsonism was rated 3 to 4 wk later using a scale from 0 (normal) to 4 (severely parkinsonian), with a maximum possible score of 28 based on seven parameters. These include spatial hypokinesia (use of available cage space), body bradykinesia (slowness in body movement), manual dexterity in both left and right hands, balance (ability to hold on to the cage bars), freezing, and action tremor as described (Quik et al. 2013b, Zhang et al. 2013, Quik et al. 2007, Quik et al. 2013a). MPTP injections were repeated (1.5-1.9 mg/kg sc per time) if the monkeys were not parkinsonian and to maintain parkinsonism. Parkinsonism was rated once weekly on Monday.

L-Dopa-Treatment and Dyskinesia Ratings.

The monkeys were next orally gavaged with L-dopa (10 mg/kg) plus carbidopa (2.5 mg/kg), twice daily 4.5 h apart 5 d per wk, as described (Quik et al. 2013b, Zhang et al. 2013, Quik et al. 2007, Quik et al. 2013a). While treated with L-dopa, monkeys were given only fruits and vegetables in the morning to enhance L-dopa absorption. Monkey chow, as well as more fruits and vegetables, were given several hours after the second dose of L-dopa. The monkeys were taped from 8:00 to 8:30 AM, before the first dose of L-dopa to evaluate baseline movements and then from 8:30 to 12:30 PM, following the morning L-dopa dose, to evaluate dyskinesias. LIDs were rated from the video recordings for a 1 min period every 30 min. Ratings were on a scale of 0 (no dyskinesias) to 4 (severely dyskinetic) with: 1=subtle dyskinesias that were not sustained (<3 trunk movements in a row); 2=sustained dyskinesias (≧3 trunk movements in a row); 3=moderate dyskinesias that impair the ability to remain stationary; and 4=severe dyskinesias that were generalized and incapacitating (Quik et al. 2013b, Zhang et al. 2013, Quik et al. 2007, Quik et al. 2013a). The effect of ABT-126 on LIDs was tested over a 2 day period (Wednesday and Thursday) on a weekly basis.

Cognitive Testing.

To evaluate cognitive performance, an object retrieval task was used that measures a component of prefrontal cortex dependent cognitive control involving inhibition of an initial learned response after subsequent spatial reversals (Lyons et al. 2000, Lyons et al. 2004, Zhang et al. 2013). The apparatus used consists of a clear Plexiglas box (8×8×8 cm) with one open side, containing a small piece of marshmallow (Lyons et al. 2000, Lyons et al. 2004). The box is locked into place on a metallic platform hooked onto the front of the monkey cage. Control and MPTP-lesioned monkeys were scored for the average time and number of trials required to retrieve the marshmallow in 30 sec. The effect of ABT-126 administration was tested once weekly on Friday.

nAChR Drug Treatments.

All monkeys were lesioned with MPTP and then gavaged with L-dopa (n=21); they thus all exhibited LIDs before the start of ABT-126 treatment. The groups were as follows: vehicle-treated (n=6), nicotine-treated (n=5), ABT-126-treated (set 1, n=5 and ABT-126-treated (set 2, n=5). These latter two sets of monkeys had previously been given ABT-894 and ABT-107 but using somewhat different treatment regimens. The present study was done after a 7 wk washout period, at which point LIDs were similar in all groups. ABT-126 was administered orally in a small cracker 30 min before L-dopa/carbidopa twice daily. Nicotine, included as a positive control. It was provided in the drinking water starting at 50 μg/ml for 2 days, increased to 150 μg/ml for another two days and then maintained at 300 μg/ml. ABT-126 was tested at 0.03, 0.10, 0.30 and 1.0 mg/kg, with each dose of ABT-126 tested for 1 or 2 wk.

ABT-126 has been discontinued and a drug washout in progress (6 wk) to determine the time required for LIDs to return to control levels.

Statistics.

Statistical analyses were done using GraphPad Prism. One- or two-way ANOVA was used for multiple group comparisons followed by a Newman-Keuls or Bonferroni post hoc test. Differences in rating scores between groups were analyzed using nonparametric tests (Mann-Whitney test). Data for rating scores was provided as the median (nonparametric) or mean±SEM (parametric). A value of P ≦0.05 is considered statistically significant. Values expressed are the mean±SEM of the indicated number of monkeys.

TABLE 1
ABT-126 was administered orally in cracker 30 min before gavage with L-
dopa (10 mg/kg)/carbidopa (2.5 mg/kg). Parkinsonism was measured
45 min before L-dopa and again 90 min after L-dopa administration
once weekly. Drug doses were 300 μg/ml nicotine in the drinking water
and 0.03 mg/kg ABT-126 orally. Values represent the mean ± SEM
of the indicated number of monkeys.

| | | Parkinsonian scores | |
|---|---|---|---|
| Group | # Monkeys | Before L-dopa | After L-dopa |
| Vehicle | 6 | 4.5 ± 0.9 | 2.3 ± 0.3 |
| Nicotine | 5 | 3.6 ± 0.4 | 2.4 ± 0.4 |
| ABT-126 (set 1) | 5 | 4.2 ± 0.4 | 2.6 ± 0.4 |
| ABT-126 (set 2) | 5 | 3.4 ± 0.7 | 2.2 ± 0.2 |

TABLE 2
Lesioned monkeys were gavaged with L-dopa twice daily at a 4.5 h
interval and given vehicle or ABT-126 orally 30 min before L-dopa.
Cognitive performance was evaluated 90 min after the first L-dopa gavage
of the day once weekly. The drug doses were 300 μg/ml nicotine in the
drinking water and 0.3 mg/kg ABT-126 orally. Values are the
mean ± SEM of the indicated number of monkeys.

| Group | # Monkeys | Latency (s) | Attempts |
|---|---|---|---|
| Vehicle | 6 | 16.0 ± 3.4 | 19.4 ± 3.4 |
| Nicotine | 5 | 19.2 ± 2.2 | 23.8 ± 2.8 |
| ABT-126 (set 1) | 5 | 21.2 ± 2.8 | 22.8 ± 3.2 |
| ABT-126 (set 2) | 5 | 18.3 ± 3.3 | 18.0 ± 2.5 |

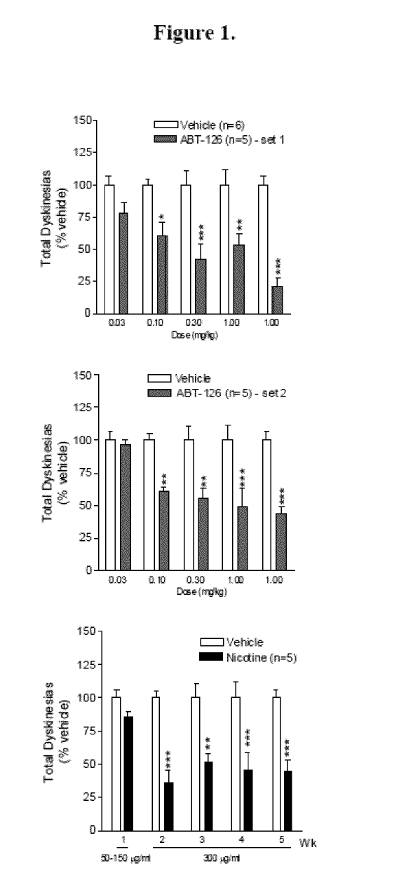

The α7 agonist ABT-126 decreases LIDs in MPTP-lesioned monkeys as shown in FIG. 1 (FIG. 1). L-dopa (10 mg/kg) plus carbidopa (2.5 mg/kg) was administered at 8:30 AM and 1 PM 5 days per wk for 5 wk, with ABT-126 given orally 30 min prior to each L-dopa gavage. The data depict the effect of increasing doses of ABT-126 (0.03 to 1.0 mg/kg) on total dyskinesia scores (expressed as % vehicle). Each dose of ABT-126 was given for an entire wk, with bars depicting the average score over 2 days. ABT-126 treatment decreased LIDs up to ˜70%. The lower panel shows the effect of nicotine (300 μg/ml in the drinking water) on LIDs in a separate group of monkeys. Values are the mean±SEM of 5-6 monkeys. Significance of difference of drug from vehicle treatment, *P<0.05, **P<0.01, ***P<using two-way ANOVA followed by a Bonferronipost hoc test.

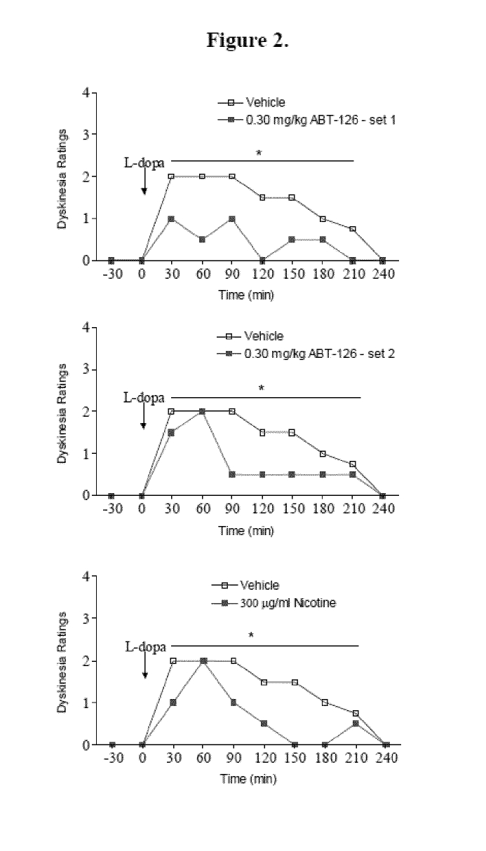

ABT-126 administration reduces the hourly time course of IDs as shown in FIG. 2 (FIG. 2). Monkeys were treated with ABT-126 as detailed in FIG. 1. ABT-126 administration reduces the hourly time course of LIDs. Monkeys were treated with ABT-126 as detailed in FIG. 1. The data in the top two panels were obtained using 0.3 mg/kg ABT-126, a dose that results in a maximal decline in LIDs. The effect of nicotine administration (300 μg/ml in the drinking water) is shown in the bottom panel. The symbol depicts the median of 5-6 monkeys. Significance of difference from vehicle using a Mann-Whitney test, *P<0.05.

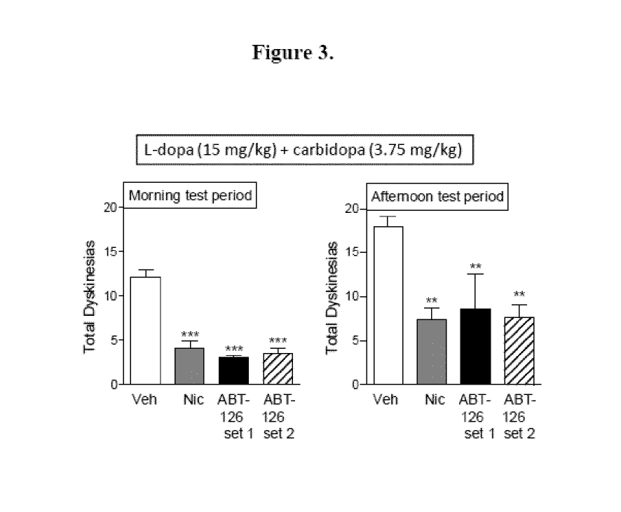

ABT-126 reduces LIDs to a similar extent with morning or afternoon L-dopa treatment as shown in FIG. 3 (FIG. 3). ABT-126 reduces LIDs to a similar extent with morning or afternoon L-dopa treatment. These studies were done at wk 6, one wk after the experiments depicted in FIG. 1. L-dopa was administered at 8:30 AM and 1 PM, with ABT-126 (1.0 mg/kg) given orally 30 min prior to each L-dopa gavage. ABT-126 was given for an entire wk, with bars depicting the average score over 2 days. The dose of L-dopa/carbidopa was increased to 15 mg/kg/3.75 mg/kg during this wk. This higher dose of L-dopa increased LIDs scores as expected. Notably, however, ABT-126 treatment still led to a 70% decline in LIDs with the morning L-dopa dose and a 60% decline with the afternoon dose, Values represent the mean±SEM of 4-6 monkeys. Significance of difference from vehicle treatment, **P<0.01, ***P<0.001 using one-way ANOVA followed by a Newman-Keuls post hoc test.

Based on the data available for Compound A, it is anticipated that doses of 50 mg QD and 75 mg QD will demonstrate efficacy in the tested subjects as effectively or more effectively than a 25 mg QD dose of Compound A.

In summary, Compound A has demonstrated a signal for efficacy in the symptomatic treatment of AD in the Phase 2a study and appears to be well tolerated in subjects with schizophrenia in doses up to 25 mg QD, including 10 mg QD and 25 mg QD, and can be anticipated to demonstrate efficacy in improving cognitive deficits of schizophrenia at doses of 50 mg QD and 75 mg QD.

It is understood that the foregoing detailed description and accompanying examples are merely illustrative and are not to be taken as limitations upon the scope of the invention, which is defined solely by the appended claims and their equivalents.

What is claimed is:

1. Use of a selective agonist of neuronal nicotinic acetylcholine receptor α7 subtype for the preparation of a medicament for treating, preventing or delaying progression of dyskinesia associated with dopamine agonist therapy in Parkinson's disease.

2. The use of claim 1, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane.

3. The use of claim 1, wherein the selective agonist of neuronal nicotinic acetylcholine receptor α7 subtype is administered in an amount of from about 6 mg to about 150 mg to a patient.

4. The use of claim 1, wherein the selective agonist of neuronal nicotinic acetylcholine receptor α7 subtype is administered in an amount of from about 10 mg to about 75 mg to a patient.

5. The use of claim 1, wherein the selective agonist of neuronal nicotinic acetylcholine receptor α7 subtype is administered in an amount of 10, 25, 50, or 75 mg to a patient once a day.

6. A method for treating, preventing or delaying progression of dyskinesia associated with dopamine agonist therapy in Parkinson's disease, comprising the step of administering a therapeutically effective amount of a selective agonist of neuronal nicotinic acetylcholine receptor α7 subtype, or a pharmaceutically acceptable salt thereof, to a patient in need of such treatment, wherein the patient is a nonsmoker.

7. The method of claim 6, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, (N-[(3R)-1-azabicyclo[2.2.2]oct-3-yl]-7-chloro-1-benzothiophene-2-carboxamide), (N-[2-(pyridin-3-ylmethyl)-1-azabicyclo[2.2.2]oct-3-yl]-1-benzofuran-2-carboxamide) or a salt thereof.

8. The method of claim 7, wherein the selective agonist of neuronal nicotinic acetylcholine receptor α7 subtype is administered in an amount of from about 10 mg to about 75 mg to a nonsmoker patient.

9. The method of claim 8, wherein the selective agonist of neuronal nicotinic acetylcholine receptor α7 subtype is administered in an amount of 10, 25, 50, or 75 mg to a nonsmoker patient once daily.

10. A pharmaceutical composition for use in the treating, preventing or delaying progression of dyskinesia associated with dopamine agonist therapy in Parkinson's disease, comprising administering a therapeutically effective amount of a selective agonist of neuronal nicotinic acetylcholine receptor α7 subtype and a pharmaceutically acceptable excipient to a patient in need of treatment.

11. The composition of claim 10, further comprising a pharmaceutically acceptable hydrophilic polymer and a pharmaceutically acceptable surfactant.

12. The composition of claim 11, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, (N-[(3R)-1-azabicyclo[2.2.2]oct-3-yl]-7-chloro-1-benzothiophene-2-carboxamide) or (N-[2-(pyridin-3-ylmethyl)-1-azabicyclo[2.2.2]oct-3-yl]-1-benzofuran-2-carboxamide) or a salt thereof.

13. The composition of claim 12, wherein the selective agonist of neuronal nicotinic acetylcholine receptor α7 subtype is in an amount of from about 25 mg to about 75 mg.

14. The composition of claim 12, wherein the selective agonist of neuronal nicotinic acetylcholine receptor α7 subtype is in an amount of from about 25, 50, or 75 mg.

15. A method for improving therapeutic efficacy of a selective agonist of neuronal nicotinic acetylcholine receptor α7 subtype, comprising:
(a) identifying a subject in need of treatment for dyskinesia associated with dopamine agonist therapy in Parkinson's disease; and
(b) administering a selective agonist of neuronal nicotinic acetylcholine receptor α7 subtype in a therapeutically effective amount to the patient in need of treatment.

16. The method of claim 15, wherein the selective agonist of neuronal nicotinic acetylcholine receptor α7 subtype is (4s)-4-(5-phenyl-1,3,4-thiadiazol-2-yloxy)-1-azatricyclo[3.3.1.1^{3,7}]decane, (N-[(3R)-1-azabicyclo[2.2.2]oct-3-yl]-7-chloro-1-benzothiophene-2-carboxamide), (N-[2-(pyridin-3-ylmethyl)-1-azabicyclo[2.2.2]oct-3-yl]-1-benzofuran-2-carboxamide) or a salt thereof.

17. Use according to claim 1, wherein the dopamine agonist therapy comprises the administration of levodopa.

18. The method according to claim 6, wherein the dopamine agonist therapy comprises the administration of levodopa.
